(19) United States
(12) Patent Application Publication
(10) Pub. No.: US 2017/0000743 A1
(43) Pub. Date: Jan. 5, 2017

(54) Compositions and Methods for Delivery of Gene Editing Tools Using Polymeric Vesicles

(71) Applicant: VINDICO NANOBIO TECHNOLOGY INC., Lexington, KY (US)

(72) Inventors: P. Peter Ghoroghchian, Boston, MA (US); Jivan Namdeo Yewle, Lexington, KY (US)

(21) Appl. No.: 15/199,021

(22) Filed: Jun. 30, 2016

Related U.S. Application Data
(60) Provisional application No. 62/187,942, filed on Jul. 2, 2015.
(60) Provisional application No. 62/322,346, filed on Apr. 14, 2016.

(51) Int. Cl. A61K 9/50; A61K 48/00; C12N 15/90

(52) U.S. Cl. CPC A61K 9/5031; C12N 15/907; A61K 48/005

(57) ABSTRACT

A composition for genetic modification and a method of forming the composition, the composition may include a synthetic polymer vesicle, and a gene editing system encapsulated in the synthetic polymer vesicle. The gene editing system may include a protein component and a nucleic acid component configured to interact with a target sequence in a host cell genome.

### RELATED APPLICATIONS

This application claims the benefit of priority to U.S. Provisional Patent Application Ser. No. 62/187,942, filed on Jul. 2, 2015, and U.S. Provisional Patent Application No. 62/322,346, filed on Apr. 14, 2016, both of which are hereby incorporated by reference in their entireties.

### BACKGROUND

A new era for genome editing technologies has recently emerged based on the development of sequence-specific nucleases. In particular, such nucleases may be used to generate DNA double strand breaks (DSBs) in precise genomic locations, and cellular repair machinery then exploited to silence or replace nucleotides and/or genes. Targeted editing of nucleic acid sequences is a highly promising approach for the study of gene function and also has the potential to provide new therapies for human genetic diseases.

Current gene editing tools include, for example, the RNA-guided DNA endonuclease Cas9, which effects sequence-specific DNA cleavage in a genome. The possibility to direct Cas9 and other enzymes to any sequence by providing specific guide RNA, and introduce controlled DNA breaks, offers a strong tool for potentially modifying the genome in vivo in a variety of ways. In particular, breaks in the DNA may result in mutation of the DNA at the cleavage site via non-homologous end joining (NHEJ) or replacement of the DNA surrounding the cleavage site via homology-directed repair (HDR), using a DNA template. The HDR template may be designed to supply a desired genetic change to a DNA sequence targeted by the nuclease.

Therefore these tools provide the potential, for example, to remove, replace, or add nucleotide bases to native DNA in order to correct or induce a point mutation, as well as to change a nucleotide base in order to correct or induce a frame shift mutation. Further, such tools may enable removing, inserting or modifying pieces of DNA containing a plurality of codons as part of one or more gene.

Currently, mechanisms for delivering nucleic acids to target cells include using viral vectors. However, viral-based gene delivery has limitations including toxicity, aggregation of the DNA or RNA, payload size limits, and difficulties with large-scale production, including costs and time.

Progress has been made in the delivery of functional DNA and RNA, using both viral vectors (e.g., retrovirus, adenovirus, etc.) and non-viral vectors. For example, wild-type AAV has attracted considerable interest from gene therapy researchers due to a number of features, such as the virus's apparent lack of pathogenicity. It can also infect non-dividing cells and has the ability to stably integrate into the host cell genome at a specific site (designated AAVS1) in the human chromosome 19. The feature makes it somewhat more predictable than retroviruses, which present the threat of a random insertion and of mutagenesis, which is sometimes followed by development of a cancer. AAV-based gene therapy vectors form episomal concatemers in the host cell nucleus. In non-dividing cells, these concatemers remain intact for the life of the host cell. In dividing cells, AAV DNA is lost through cell division, since the episomal DNA is not replicated along with the host cell DNA. Random integration of AAV DNA into the host genome is detectable but occurs at very low frequency. AAVs also present very low immunogenicity, seemingly restricted to generation of neutralizing antibodies, while they induce no clearly defined cytotoxic response. These features, along with the ability to infect quiescent cells, demonstrate that AAVs are dominant over adenoviruses as vectors for human gene therapy. However, the use of viral vectors (including AAVs) is also associated with some disadvantages, in particular the limited size of viral genomes. For example, the AAV genome is only 4.8 kilobase (kb), and therefore is unable to be used for single-vehicle delivery of the multitude of gene editing tools of the various embodiments.

Further drawbacks to the use of viruses to deliver gene editing tools may include targeting only dividing cells, random insertion into the host genome, risk of replication, and possible host immune reaction, as well as limitations on payload size imposed by the viral capsid, which in particular prevents incorporation of a multitude of different gene editing tools that are required to achieve site-specific gene correction.

In general, non-viral vectors are typically easy to manufacture, less likely to produce immune reactions, and do not produce replication reactions compared to viral vectors; existing methods are generally ineffective for in vivo introduction of genetic material into cells and have resulted in relatively low gene expression. Specifically, a number of existing non-viral systems have been recently explored for delivery of gene editing tools in the form of proteins and/or nucleic acids to cells. Such system may be broadly classified as “nanocapsules” in which a slurry of free DNA/RNA/protein is wrapped with polymer peptide; “lipid-based vehicles” (e.g., liposomes, lipid-based nanoparticles, etc.) modified with cationic amphiphilic polymers to self assemble with the nucleic acids based on charge; and “bioconjugates” (e.g., lipids, synthetic macromolecules, etc.) that target the nucleic acid, including via binding to specific proteins expressed by target cells to enable cellular internalization. Each of these non-viral systems presents its own set of issues with respect to encapsulating either single or a multitude of gene editing tools in a single delivery vehicle. For example, in a nanocapsule system, the structure is highly unstable and may leak its contents into the vasculature after intravenous administration. As such, the capability to achieve intracellular delivery and release of a sufficient quantity of material components necessary for effective gene editing is unlikely. In lipid-based vehicles, the charged delivery systems have demonstrated poor loading capacity and difficult release of encapsulated payload. In a bioconjugate system, the use of a vector of sufficient size will expose the nucleic acids directly to nucleases in the blood stream/cytosol that will cause fragmentation and destruction of the payload, obviating the ability to achieve efficient gene editing.

Therefore, an effective vehicle for delivering nucleic acids, such as small guide RNA, nucleic acids such as mRNA, and/or large DNA plasmids, to target cells, as well as proteins such as Cas9, is needed.

### SUMMARY

Systems and methods enable genetic modification through a composition that includes a synthetic polymer vesicle, and a gene editing system encapsulated in the synthetic polymer vesicle. In some embodiments, the gene editing system may include a nucleic acid component configured to interact with a target sequence in a host cell genome. In some embodiments, the gene editing system may also include a protein component. In some embodiments, the protein component may include an RNA-directed nuclease, and the nucleic acid component may include a guide ribonucleic acid (RNA) that is specific to the target sequence. In some embodiments, the nucleic acid component may also include a deoxyribonucleic acid (DNA) repair template. In some embodiments, the RNA-directed nuclease may create at least one break in the host cell genome, and a repair process in the host cell may trigger modification of at least one nucleotide in the host cell genome based on the exogenous DNA repair template, in which the at least one nucleotide is incorporated into re-ligation of the host cell genome.

In some embodiments, the DNA repair process may be initiated based on regions on the DNA repair template that are homologous to regions on either side of a double stranded break in the target sequence induced by the RNA-directed nuclease. In some embodiments, the gene editing system may be included in the synthetic polymer vesicle in an amount of at least 3% by weight relative to the total weight of the composition.

In some embodiments, the protein component may include an enzyme in a native form. In some embodiments, protein component may include a messenger RNA (mRNA) molecule that is translated into an enzyme after delivery into the host cell. In some embodiments, the protein component may be as an expression vector containing a deoxyribonucleic acid (DNA) sequence encoding one or more gene to express an enzyme. In some embodiments, the nucleic acid component may be a DNA sequence encoding one or more gene to express a guide ribonucleic acid (RNA).

In some embodiments, the DNA sequence encoding the gene to express the enzyme and the guide RNA may be provided on a single expression vector. In some embodiments, the protein component may include an enzyme configured to cut the host genome based on binding of the nucleic acid component to a segment of the host genome. In some embodiments, the enzyme is the Clustered Regularly Interspaced Short Palindromic Repeats (CRISPR) associated protein 9 (Cas9). In some embodiments, the nucleic acid component includes an expression vector that includes at least one transposon, and the protein component includes a transposase agent. In some embodiments, the transposase agent may be one of a native enzyme, a messenger ribonucleic acid (mRNA) molecule that is translated into the enzyme after delivery into the host cell, and a deoxyribonucleic acid (DNA) sequence encoding one or more gene to express the enzyme. In some embodiments, the DNA sequence encoding the gene to express the enzyme may be provided on the expression vector that includes the transposon.

In some embodiments, the synthetic polymer vesicle may be generated from at least one block copolymer that includes a hydrophilic block containing poly(ethylene oxide), and a hydrophobic block. In some embodiments, the hydrophobic block may be selected from aliphatic poly(anhydrides), poly(nucleic acids), poly(esters), poly(ortho esters), poly(peptides), poly(phosphazenes) and poly(saccharides). In some embodiments, the hydrophobic block may comprise one or more of poly(lactide) (PLA), poly(glycolide) (PLGA), poly(lactic-co-glycolic acid) (PLGA), poly(ε-caprolactone) (PCL), or poly (trimethylene carbonate) (PTMC).

Systems and methods for modifying a host cell genome in various embodiments may include encapsulating, in a polymersome, a gene editing system that includes a protein component and a nucleic acid component, and delivering the encapsulated gene editing system to the host cell. In some embodiments, the nucleic acid component may be configured to interact with a target nucleic acid sequence in the host cell. In some embodiments, the polymersome is configured to selectively release the gene editing system in the host cell.

Embodiment methods may further include delivering the encapsulated gene editing system to the host cell by administering to a subject an effective amount of a composition containing the encapsulated gene editing system. In some embodiments, the encapsulated gene editing system may be prepared using a progressive saturation protocol.

Systems and methods of manufacturing a suspension of an encapsulated gene editing composition in various embodiments may include thermally blending a quantity of a block copolymer with a quantity of a low molecular weight polyethylene glycol (PEG) to create a PEG/polymer formulation, adding an aliquot of a solution of the gene editing composition to a sample containing the PEG/polymer formulation, and performing at least one dilution step such that polymersomes that are generated are progressively saturated with the gene editing composition. In some embodiments, the gene editing composition may include a protein component and a nucleic acid component configured to interact with a target sequence in a host cell genome.

In some embodiments, the block copolymer may include an amphiphilic diblock copolymer. In some embodiments, the amphiphilic diblock copolymer may include poly(ethylene oxide)-block-poly(butadiene) (PEO-b-PBD). In some embodiments, the generated polymersomes may have an encapsulation efficiency of at least 50% for the gene editing composition.

A system in various embodiments may be implemented as a kit that includes a pharmaceutical composition having a gene editing system encapsulated in a synthetic polymer vesicle, and an implement for administering the pharmaceutical composition intravenously, via inhalation, topically, per rectum, per the vagina, transdermally, subcutaneously, intraperitoneally, intrathecally, intramuscularly, or orally. In some embodiments, the gene editing system may include a protein component and a nucleic acid component configured to interact with a target sequence in a host cell genome.

### BRIEF DESCRIPTION OF THE DRAWINGS

The accompanying drawings, which are incorporated herein and constitute part of this specification, illustrate exemplary embodiments, and together with the descriptions of various embodiments, serve to explain the features herein.

FIG. 1 is a table of properties for two poly(ethylene oxide)-block-poly(butadiene) (i.e., PEO-b-PBD) diblock copolymers and their polymersome formulations used for various nanoparticle encapsulations.

FIGS. 2A and 2B are graphs showing co-encapsulation of a model protein (myoglobin) or plasmid DNA encoding the mammalian DNA vector for expression of green fluorescent protein (GFP) using the elongation factor 1 alpha (EF1a) promoter) (i.e., pEF-GFP DNA) into polymersomes formed from a particular PEO-b-PBD formulation resulting from use of the progressive saturation protocol.

FIGS. 2C through 2E are graphs showing co-encapsulation of a model protein (myoglobin) and plasmid DNA encoding the mammalian expression vector for expression of green fluorescent protein (GFP) using the elongation factor 1 alpha (EF1a) promoter) (i.e., pEF-GFP DNA) into the same polymersome construct formed from a particular PEO-b-PBD formulation resulting from use of a progressive saturation protocol.

FIGS. 3A through 3E are graphs showing co-encapsulation of the model protein (myoglobin) and pEF-GFP DNA into the same polymersome construct formed from a particular PEO-b-PBD formulation resulting from use of the thin film hydration protocol.

FIGS. 4A through 4E are graphs showing co-encapsulation of the model protein bovine serum albumin (BSA) and pEF-GFP DNA into the same polymersome construct formed from a particular PEO-b-PBD formulation resulting from use of the progressive saturation protocol.

FIGS. 5A through 5C are graphs showing encapsulation of the functional Cas9 protein from Streptococcus pyogenes into polymersomes formed from a particular PEO-b-PBD formulation resulting from use of the progressive saturation protocol.

FIGS. 6A through 6E are graphs showing co-encapsulation of the functional Cas9 protein from Streptococcus pyogenes and pEF-GFP DNA into the same polymersome constructs formed from a particular PEO-b-PBD formulation resulting from use of the progressive saturation protocol.

### DETAILED DESCRIPTION

An embodiment composition for genetic modification may be prepared to include a synthetic polymer vesicle, and a gene editing system encapsulated in the synthetic polymer vesicle in which the gene editing system includes a nucleic acid component configured to interact with a target sequence in a host cell genome. In an embodiment, the gene editing system may also include a protein component. In an embodiment, the protein component may be a ribonucleic acid (RNA)-directed nuclease, and the nucleic acid component may be a guide RNA that is complementary to the target sequence. In an embodiment, the nucleic acid component may also include an exogenous deoxyribonucleic acid (DNA) repair template, and the RNA-directed nuclease may be configured to create a double stranded break in the host cell genome adjacent to the target sequence. In an embodiment, a repair process in the host cell may trigger modification of the host cell genome based on the exogenous DNA repair template, during re-ligation of the host cell genome. In an embodiment, the DNA repair template may have end regions that are homologous to regions of the host cell genome flanking the double stranded break induced by the RNA-directed nuclease. In an embodiment, the gene editing system may be included in the synthetic polymer vesicle in an amount of at least 3% by weight relative to the total weight of the composition. In an embodiment, the protein component may be an enzyme in native form or a messenger RNA (mRNA) molecule configured to be translated into an enzyme after delivery into the host cell. In an embodiment, the protein component may be delivered as an expression vector containing a deoxyribonucleic acid (DNA) sequence encoding an enzyme. In an embodiment, the nucleic acid component may be a DNA sequence encoding a guide ribonucleic acid (RNA). In an embodiment, the DNA sequence encoding the enzyme and the guide RNA may be provided on a single expression vector. In an embodiment, the protein component may be an enzyme configured to cut the host genome based on binding of the nucleic acid component to a complementary segment of the host genome. In an embodiment, the enzyme may be a Clustered Regularly Interspaced Short Palindromic Repeats (CRISPR) associated protein 9 (Cas9). In an embodiment, the nucleic acid component may be an expression vector that includes a transposon, and the protein component may be a transposase. In an embodiment, the transposase may be one of a native enzyme, a messenger ribonucleic acid (mRNA) molecule that is configured to be translated into the enzyme after delivery into the host cell, and a DNA sequence encoding the enzyme. In an embodiment, the DNA sequence may be provided on the expression vector that includes the transposon. In an embodiment, the synthetic polymer vesicle may be generated from at least one block copolymer that includes a hydrophilic block with poly(ethylene oxide), and a hydrophobic block. In an embodiment, the hydrophobic block may be selected from aliphatic poly(anhydrides), poly(nucleic acids), poly(esters), poly(ortho esters), poly(peptides), poly(phosphazenes) and poly(saccharides). In an embodiment, the hydrophobic block may include one or more of poly(lactide) (PLA), poly(glycolide) (PLGA), poly(lactic-co-glycolic acid) (PLGA), poly(ε-caprolactone) (PCL), or poly (trimethylene carbonate) (PTMC).

In an embodiment, a method of modifying a host cell genome may include encapsulating, in a polymersome, a gene editing system, and delivering the encapsulated gene editing system to the host cell, in which the polymersome is configured to selectively release the gene editing system in the host cell. In an embodiment, the gene editing system may include a protein component, and a nucleic acid component configured to interact with a target nucleic acid sequence in the host cell. In an embodiment, delivering the encapsulated gene editing system to the host cell may be performed by administering to a subject an effective amount of a composition containing the encapsulated gene editing system. In an embodiment, the encapsulated gene editing system may be prepared using a progressive saturation protocol. In an embodiment, manufacturing a suspension of an encapsulated gene editing composition may include thermally blending a quantity of a block copolymer with a quantity of a low molecular weight polyethylene glycol (PEG) to create a PEG/polymer formulation, adding an aliquot of a solution of the gene editing composition to a sample containing the PEG/polymer formulation, and performing at least one dilution step such that polymersomes that are generated are progressively saturated with the gene editing composition. In an embodiment, the gene editing composition may include a protein component, and a nucleic acid component configured to interact with a target sequence in a host cell genome. In an embodiment, the block copolymer may be an amphiphilic diblock copolymer. In an embodiment, the amphiphilic diblock copolymer, may be poly(ethylene oxide)-block-poly(butadiene) (PEO-b-PBD). In an embodiment, the generated polymersomes have an encapsulation efficiency of at least 50% with respect to the gene editing composition.

The various embodiments will be described in detail with reference to the accompanying drawings. Wherever possible, the same reference numbers will be used throughout the drawings to refer to the same or like parts. References made to particular examples and implementations are for illustrative purposes, and are not intended to limit the scope of the claims.

It is to be appreciated that certain features that are, for clarity, described herein in the context of separate embodiments, may also be provided in combination in a single embodiment. Conversely, various features that are, for brevity, described in the context of a single embodiment, may also be provided separately or in any sub-combination. Further, reference to values stated in ranges includes each and every value within that range.

As used in this specification and the appended claims, the singular forms “a,” “an,” and “the” include plural referents unless the content clearly dictates otherwise.

The word “plurality” is used herein to mean more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

The terms “subject” and “patient” are used interchangeably herein to refer to human patients, whereas the term “subject” may also refer to any animal. It should be understood that in various embodiments, the subject may be a mammal, a non-human animal, a canine and/or a vertebrate.

The term “monomeric units” is used herein to mean a unit of polymer molecule containing the same or similar number of atoms as one of the monomers. Monomeric units, as used in this specification, may be of a single type (homogeneous) or a variety of types (heterogeneous).

The term “polymer” is used according to its ordinary meaning of a macromolecule comprising connected monomeric molecules.

The term “amphiphilic” is used herein to mean a substance containing both polar (water-soluble) and hydrophobic (water-insoluble) groups.

The term “an effective amount” is used herein to refer to an amount of a compound, material, or composition effective to achieve a particular biological result such as, but not limited to, biological results disclosed, described, or exemplified herein. Such results may include, but are not limited to, the effective reduction of symptoms associated with any of the disease states mentioned herein, as determined by any means suitable in the art. As recognized by those of ordinary skill in the art, the effective amount of an agent, e.g., a nuclease, an integrase, a transposase, a recombinase, a hybrid protein, a fusion protein, a protein dimer, a complex of a protein (or protein dimer) and a polynucleotide, or a polynucleotide, may vary depending on various factors as, for example, on the desired biological response, the specific allele, genome, target site, cell, or tissue being targeted, and the agent being used.

The term “membrane” is used herein to mean a spatially distinct collection of molecules that defines a two-dimensional surface in three-dimensional space, and thus separates one space from another in at least a local sense.

The term “active agent” is used herein to refer to any a protein, peptide, sugar, saccharide, nucleoside, inorganic compound, lipid, nucleic acid, small synthetic chemical compound, or organic compound that appreciably alters or affects the biological system to which it is introduced.

The term, “vehicle” is used herein to refer to agents with no inherent therapeutic benefit but when combined with an active agent for the purposes of delivery into a cell result in modification of the active agent's properties, including but not limited to its mechanism or mode of in vivo delivery, its concentration, bioavailability, absorption, distribution and elimination for the benefit of improving product efficacy and safety, as well as patient convenience and compliance.

The term “carrier” is used herein to describe a delivery vehicle that is used to incorporate a pharmaceutically active agent for the purposes of drug delivery.

The term “homopolymer” is used herein to refer to a polymer derived from one monomeric species of polymer.

The term “copolymer” is used herein to refer to a polymer derived from two (or more) monomeric species of polymer, as opposed to a homopolymer where only one monomer is used. Since a copolymer consists of at least two types of constituent units (also structural units), copolymers may be classified based on how these units are arranged along the chain.

The term “block copolymers” is used herein to refer to a copolymer that includes two or more homopolymer subunits linked by covalent bonds in which the union of the homopolymer subunits may require an intermediate non-repeating subunit, known as a junction block. Block copolymers with two or three distinct blocks are referred to herein as “diblock copolymers” and “triblock copolymers,” respectively.

The term “loading capacity” is used herein to refer to the weight of a particular compound within a carrier divided by the total weight of carrier. The terms “encapsulation efficiency” and “loading efficiency” are interchangeably used herein to refer to the weight a particular compound that is encapsulated and/or incorporated within a carrier suspension divided by the weight of the original compound in solution prior to encapsulation (expressed as a %).

The terms “nucleic acid” and “nucleic acid component” are used interchangeably herein to refer to a compound with a nucleobase and an acidic moiety, e.g., a nucleoside, a nucleotide, or a polymer of nucleotides. Typically, polymeric nucleic acids, e.g., nucleic acid components comprising three or more nucleotides are linear molecules, in which adjacent nucleotides are linked to each other via a phosphodiester or a phosphorothioate linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid component. On the other hand, a nucleic acid component may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone including a phosphorothioate linkage.

Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The terms “RNA-guided nuclease” and “RNA-guided endonuclease” are used interchangeably herein and refer to a nuclease that forms a complex with (e.g., binds or associates with) one or more RNA that is not a target for cleavage. An example RNA-guided nuclease is the RNA-guided nuclease is the (CRISPR-associated system) Cas9 endonuclease. In some embodiments, an RNA-guided nuclease, when in a complex with an RNA, may be referred to as a nuclease:RNA complex. Typically, the bound RNA(s) is referred to as a guide RNA. Guide RNAs may exist as a complex of two or more RNAs, or as a single RNA molecule. While guide RNAs that exist as a single RNA molecule may be referred to as single-guide RNAs (sgRNAs), the terms “guide RNA” and “gRNA” may be used interchangeably herein to refer to guide RNAs that exist as either single molecules or as a complex of two or more molecules. Typically, guide RNAs that exist as single RNA species comprise two domains: (1) a domain that shares homology to a target nucleic acid (e.g., and directs binding of a Cas9 complex to the target); and (2) a domain that binds a Cas9 protein. In some embodiments, domain (2) corresponds to a sequence known as a tracrRNA, and comprises a stem-loop structure. For example, in some embodiments, domain (2) is homologous to a tracrRNA. The guide RNA comprises a nucleotide sequence that complements a target site, which mediates binding of the nuclease/RNA complex to the target site, providing the sequence specificity of the nuclease:RNA complex. Because RNA-guided nucleases (e.g., Cas9) use RNA:DNA hybridization to target DNA cleavage sites, these proteins are able to be targeted, in principle, to any sequence specified by the guide RNA.

The terms “treatment,” “treat,” and “treating,” refer to a clinical intervention aimed to reverse, alleviate, delay the onset of, or inhibit the progress of a disease or disorder, or one or more symptoms thereof, as described herein. As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

The correct and efficient repair of double-strand breaks (DSBs) in DNA is critical to maintaining genome stability in cells. Structural damage to DNA may occur randomly and unpredictably in the genome due to any of a number of intracellular factors (e.g., nucleases, reactive oxygen species, etc.) as well as external forces (e.g., ionizing radiation, ultraviolet (UV) radiation, etc.). In particular, correct and efficient repair of double-strand breaks (DSBs) in DNA is critical to maintaining genome stability. Accordingly, cells naturally possess a number of DNA repair mechanisms, which can be leveraged to alter DNA sequences through controlled DSBs at specific sites. Genetic modification tools may therefore be composed of programmable, sequence-specific DNA-binding modules associated with a nonspecific DNA nuclease, introducing DSBs into the genome. For example CRISPR, mostly found in bacteria, are loci containing short direct repeats, and are part of the acquired prokaryotic immune system, conferring resistance to exogenous sequences such as plasmids and phages. RNA-guided endonucleases are programmable genetic engineering tools that are adapted from the CRISPR/CRISPR-associated protein 9 (Cas9) system, which is a component of prokaryotic innate immunity.

In eukaryotic cells, mechanistic repair of DSBs occurs primarily by two pathways: Non-Homologous End-Joining (NHEJ) and Homology Directed Repair (HDR). NHEJ is a homology-independent pathway that requires alignment of only one or a few complementary bases at the re-ligation of two ends. HDR, which is typically a more accurate mechanism for DSB repair, uses longer stretches of sequence homology to repair DNA breaks. Specifically, various HDR pathways are characterized by the use of a homologous donor (e.g., sister chromatid, plasmid, oligonucleotide (i.e., oligo-DNA), etc.). In HDR, DSB repair involves resecting the 5′-ended DNA strand at the break to create a 3′ overhang Subsequently, the 3′ single-stranded DNA (i.e., the 3′ overhang) may invade into a homologous DNA duplex, displacing one strand and pairing with the other to create a displacement loop (D-loop) structure consisting of a region of heteroduplex DNA and displaced single strand of DNA. Finally, the recombination intermediates are resolved to complete the DNA repair.

Two particular HDR pathways that offer different resolutions to complete the repair include double-strand break repair (DSBR) and synthesis-dependent strand-annealing (SDSA). In the DSBR pathway, the 3′ overhangs invade an intact homologous template and serve as a primer for DNA repair synthesis, The D-loop can be extended by the initiation of new DNA synthesis from the 3′ end of the invading strand or the action of helicases, so that the 3′ overhang of the opposite side of the DSB can anneal, thus forming a double “Holliday junction” (dHJ) intermediate. Alternatively, two independent strand invasions from both DSB ends, followed by simultaneous DNA synthesis and annealing could also result in a dHJ intermediate. Experimental evidence is unclear as to whether recombination depends on one end or whether both undergo strand invasion. These dHJs can be cleaved by one of several HJ resolvases, and, depending on which pair of strands is cut, can yield a non-crossover (i.e., all newly synthesized sequences on same molecule) or crossover (combination of new and old sequences on each molecule) outcome. As an alternative to cleavage, dHJs can be “dissolved” to yield exclusively a non-crossover outcome.

The SDSA pathway is conservative, and results exclusively in non-crossover events. In the SDSA pathway, following strand invasion and D-loop formation in SDSA, the newly synthesized portion of the invasive strand is displaced from the template and returned to the processed end of the non-invading strand at the other DSB end. The 3′ end of the non-invasive strand is elongated and ligated to fill the gap, thus completing SDSA.

Polymersomes are synthetic polymer vesicles that are formed in nanometric dimensions (50 to 300 nm in diameter) and exhibit several favorable properties as cellular oxygen carriers. For example, polymersomes belong to the class of bi- and multi-layered vesicles that can be generated through self-assembly and can encapsulate hydrophilic compounds such as hemoglobin (Hb) and myoglobin (Mb) in their aqueous core. Moreover, polymersomes offer several options to be designed from fully biodegradable FDA-approved components and exhibit no in vitro or acute in vivo toxicities.

Polymersomes exhibit several superior properties over liposomes and other nanoparticle-based delivery vehicles that make them effective carriers for various molecules. For example, depending on the structure of their component copolymer blocks, polymersome membranes may be significantly thicker (˜9-22 nm) than those of liposomes (3-4 nm), making them 5-50 times mechanically tougher and at least 10 times less permeable to water than liposomes. The circulatory half-life of polymersomes, with poly(ethylene oxide) (PEO) brushes ranging from 1.2-3.7 kDa, is analogous to that of poly (ethylene glycol)-based liposomes (PEG-liposomes) of similar sizes (˜24-48 hours) and can be further specifically tailored by using a variety of copolymers as composite building blocks. Polymersomes have been shown to be stable for several months in situ, and for several days in blood plasma under well-mixed quasi-physiological conditions, without experiencing any changes in vesicle size and morphology. They do not show in-surface thermal transitions up to 60° C. In addition, early animal studies on PEO-b-PCL and poly(ethylene-oxide)-block-poly(butadiene)- (PEO-b-PBD-)based polymersomes formulations encapsulating doxorubicin have shown no acute or sub-acute toxicities. Finally, the production and storage of polymersomes is economical. Polymersomes may be readily produced and stored on a large-scale without requiring costly post-manufacturing purification processes.

Promising biodegradable polymersome-encapsulated protein formulations may be comprised of block copolymers that consist of the hydrophilic biocompatible poly(ethylene oxide) (PEO), which is chemically synonymous (and used interchangeably herein) with PEG, poly(nucleic acids), poly(esters), poly(ortho esters), poly(peptides), poly(phosphazenes) and poly(saccharides), including but not limited by poly(lactide) (PLA), poly(glycolide) (PLGA), poly(lactic-co-glycolic acid) (PLGA), poly(ε-caprolactone) (PCL), and poly (trimethylene carbonate) (PTMC). Polymersomes comprised of 100% PEGylated surfaces possess improved in vitro chemical stability, augmented in vivo bioavailablity, and prolonged blood circulatory half-lives. For example, aliphatic polyesters, constituting the polymersomes' membrane portions, are degraded by hydrolysis of their ester linkages in physiological conditions such as in the human body. Because of their biodegradable nature, aliphatic polyesters have received a great deal of attention for use as implantable biomaterials in drug delivery devices, bioresorbable sutures, adhesion barriers, and as scaffolds for injury repair via tissue engineering.

In various embodiments, molecules required for gene editing (i.e., gene editing tools) may be delivered to cells using a single polymersome carrier. The term “gene editing” as used herein refers to the insertion, deletion or replacement of nucleic acids in genomic DNA so as to add, disrupt or modify the function of the product that is encoded by a gene. Various gene editing systems require, at a minimum, the introduction of a cutting enzyme (e.g., a nuclease or recombinase) that cuts genomic DNA to disrupt or activate gene function. Further, in gene editing systems that involve inserting new or existing nucleotides/nucleic acids, insertion tools (e.g. DNA template vectors, a transposon or retrotransposon) must be delivered to the cell in addition to the cutting enzyme (e.g. a nuclease, recombinase, integrase or transposase). Examples of such insertion tools for a recombinase may include a DNA vector. For a nuclease, examples of insertion tools may include a template DNA for HDR (to insert newly synthesized nucleotides); other gene editing systems require the delivery of an integrase along with an insertion vector, a transposase along with a transposon/retrotransposon, etc. In some embodiments, an example recombinase that may be used as a cutting enzyme is the CRE recombinase. In various embodiments, example integrases that may be used in insertion tools include viral based enzymes taken from any of a number of viruses (e.g., AAV, gamma retrovirus, lentivirus, etc.). Example transposons/retrotransposons that may be used in insertion tools include piggybac, sleeping beauty, L1, etc.

In various embodiments, nucleases that may be used as cutting enzymes include, but are not limited to, Cas9, transcription activator-like effector nucleases (TALENs), and zinc finger nucleases. Embodiments in which Cas9 is the cutting enzyme also require a guide RNA, which can be delivered to the cell in the form of RNA or as part of a DNA vector that is then transcribed intracellularly. As such, an example gene editing system that uses a nuclease and DNA template for HDR requires delivery of a least two gene editing tools to the same cell (i.e., nuclease and DNA template), and three tools for the specific nuclease Cas9 (i.e., Cas9, guide RNA, and DNA template). In another example, gene editing systems that use a nuclease and a transposon with transposase require delivery of at least three gene editing tools to the same cell (i.e., nuclease, transposon, and transposase), and four tools for the specific nuclease Cas9 (i.e., Cas9, guide RNA, transposon, and transposase).

In various embodiments, the gene editing systems described herein, including those that require two or more gene editing tools, may be encapsulated in a single nanoparticle carrier. In particular, polymersome encapsulation of a set of gene editing tools may enable efficient delivery to a cell of all molecules needed to perform a desired gene modification.

The polymersome-based delivery system provides substantial flexibility with respect to materials, as well as a large payload capacity, in vivo stability, and targeted release of the nanoparticle payload. For example, the polymersomes may be configured to release the contents thereof as a result of a change in pH, such as at a pH encountered in a cellular endosome.

In one example, site-specific cleavage of the double stranded DNA may be enabled by delivery of RNA-directed nuclease and guide RNA using various polymersomes. The RNA molecule in various embodiments may be made of two noncoding RNA elements: a CRISPR RNA (crRNA) containing 20 by of a unique sequence (spacer sequence) that is complementary to, and heterodimerizes with, a target sequence in the native DNA; and (2) trans-activating crRNA (tracrRNA). The crRNA:tracrRNA duplex directs Cas9 to the target DNA in the genome via complementary base pairing between the spacer on the crRNA and the complementary sequence (protospacer) on the target DNA, creating a DNA/RNA complex. Target specificity of Cas9 protein relies on the presence of specific nucleotide bases in the opposite strand of DNA with respect to the DNA/RNA complex direction, termed the protospacer adjacent motif (PAM). For example, the Cas9 RNA-guided nuclease from Streptococcus pyogenes, spCas9, requires a 5′-NGG-3′ PAM.

The RNA-directed nuclease recognizes the RNA/DNA complex and creates a DSB within the target sequence. The DSB is located three bases from the PAM sequence on the opposite strand with respect to the DNA/RNA complex. That is, the PAM sequence (e.g., NGG) follows, in the 3′ direction, the region on the opposite strand that is complementary to the protospacer.

In some embodiments, polymersomes may be used to encapsulate RNA-directed nuclease may be in the native protein form. In some embodiments, an mRNA encoding the RNA-directed nuclease may be instead be encapsulated and, once inside the cell, translated into the amino acids that form the enzyme.

Various embodiments may be DNA-based systems that are encapsulated into polymersomes. In some embodiments, an expression vector that expresses the RNA-directed nuclease may be encapsulated in a polymersome. The expression vector may be, for example, a plasmid constructed to contain DNA encoding the RNA-directed nuclease as well as a promoter region. Once inside the target cell, the DNA encoding the RNA-directed nuclease may be transcribed and translated to create the enzyme. In some embodiments, the expression vector may be constructed to express the guide RNA as well as the RNA-directed nuclease. In various embodiments, HDR may be utilized for gene editing by also encapsulating exogenous DNA to serve as an HDR template. That is, in some embodiments, a polymersome may encapsulate a DNA repair template in addition to the RNA-guided nuclease and the guide RNA. The repair template may contain the desired sequence for gene editing, as well as additional homologous sequence immediately upstream and downstream of the target (termed left & right homology arms). The length and binding position of each homology arm is dependent on the size of the change being introduced. The repair template may be a single-stranded oligodeoxynucleotide (ssODN), double-stranded oligodeoxynucleotide (dsODN), or double-stranded DNA (dsDNA) plasmid depending on the specific application. For example, ssDNA templates (e.g., ssODNs or dsODNs) may be used to introduce small modifications (e.g., up to around 50 by or single point mutations). In various embodiments, ssODNs and dsODNs may also include at homology arms of at least 40 base pairs on either side of the intended mutation. For larger inserts, dsDNA encompassing homology arms of 800 by each or larger may be used (e.g., in a plasmid that has been linearized or as a transposon).

Thus, various guide RNA molecules may be designed to mutate, activate, or repress almost any gene using Cas9 coupled with highly specific DNA repair templates.

In various embodiments, multiplex gene editing applications may be accomplished using RNA-directed nuclease (e.g., Cas9) and multiple guide RNAs. Such applications include the use of Cas9 to generate a large genomic deletion, and/or the modification of several genes (e.g., 2-7 loci) at once. In some systems, 2-7 genetic loci may be targeted by cloning multiple gRNAs

Various embodiment systems may also be designed to integrate DNA into the genome of a target cell using a transposon provided on a vector, such as an artificially constructed plasmid. Applications of such systems may include introducing (i.e., “knocking in”) a new gene to perform a particular function through the inserted DNA, or inactivating (i.e., “knocking out”) a mutated gene that is functioning improperly through interruption in the target DNA.

In some embodiments, the DNA may be transposon that is directly transposed between vectors and chromosomes via a “cut and paste” mechanism. In some embodiments, the transposon may be a retrotransposon—that is, DNA that is first transcribed into an RNA intermediate, followed by reverse transcription into the DNA that is transposed.

In various embodiments, the polymersomes may encapsulate a vector that includes the transposon, as well a transposase that catalyzes the integration of the transposon into specific sites in the target genome. The transposase that is used is specific to the particular transposon that is selected, each of which may have particular properties are desirable for use in various embodiments. One example transposon is the piggybac transposon, which is transposed into a target genome by the piggybac transposase. Specifically, the piggybac transposase recognizes transposon-specific inverted terminal repeat sequences (ITRs) on the ends of the transposon, and moves the contents between the ITRs into TTAA chromosomal sites. The piggybac transposon system has no payload limit for the genes of interest that can be included between the ITRs. Another example transposon system is the sleepingbeauty transposon, which is transposed into the target genome by the sleepingbeauty transposase that recognizes ITRs, and moves the contents between the ITRs into TA chromosomal sites. In various embodiments, SB transposon-mediated gene transfer, or gene transfer using any of a number of similar transposons, may be used for long-term expression of a therapeutic gene.

Similar to the RNA-directed nucleases discussed here, polymersomes may encapsulate the transposase in its native protein for, as mRNA that is transcribed into protein in the target cell, or as an expression vector containing DNA to express the transposase protein. For example, genes encoding the transposase may be provided in the same vector as the transposon itself, or on a different vector.

Various embodiments may further enable encapsulating an RNA-directed nuclease, one or more guide RNA, and a transposon system in a polymersome for delivery to a target cell. Such polymersomes may be used for example, to replace a mutated gene that causes disease with a healthy copy of the gene that is inserted at a specific site dictated by the activity of the nuclease. Specifically, a transposon may be created that includes one or more gene to be inserted, which is surrounded by the ITRs for recognition by the transposase. The transposon and ITRs may be provided on a vector that contains homology arms on each end of the ITRs. The transposon system (i.e., the transposon vector and corresponding transposase), when delivered with the RNA-directed nuclease and the guide RNA, may serve the function of the DNA repair template used in HDR. That is, following the creation of one or more DSB by the RNA-guided nuclease, instead of repair through HDR or NHEJ, the transposon may be inserted into the target DNA based on the homology arms. In some embodiments, the transposon insertion may occur between the two ends generated by a DSB. In other embodiments, the transposon may be inserted between one arm of a first DSB and the other arm at a second DSB in the target DNA (i.e., replacing the sequence between two DSBs).

While a variety polymersome formulations that encapsulate proteins and/or nucleic acids may be designed for different uses, each encapsulation system may include common characteristics in order to be effective. For example, nucleic acids may be encapsulated by polymersomes with at least 50% efficiency of encapsulation, and may make up at least 10-20 wt % of the final nanoparticle formulation by weight. Such minimum weight percentage and efficiency ensures delivery of enough nucleic acid to achieve efficient DNA cleavage, and that the product can be reproducibly generated at a low cost. In another example, the polymersomes may be designed to be stable, yet to provide facile release of the encapsulated payload once the polymersome has been taken up intracellularly, thereby avoiding endosomal retrafficking and ensuring release of the nucleic acids. Moreover, in various gene therapy systems, the vector (i.e., transposon) may be designed to provide stable expression.

The gene editing tools provided in the polymersome encapsulations described herein may be beneficial for a number of in vivo applications. For example, the embodiment materials may be delivered to various cell types in order to cut or to repair gene defects. Such cells include, but are not limited to, hepatocytes, hepatic endothelial cells, immune cells, neurons, etc. The embodiment polymersomes may also be delivered to various cell types in order to silence defective genes that cause diseases (for example, delivery to retinal cells to silence mutations underlying Leber's Congenital Amaurosis).

Various methods may be used to generate the polymersome encapsulations and/or co-encapsulations of proteins and/or nucleic acids described herein. In some embodiments, conventional encapsulation techniques such as thin-film rehydration, direct-hydration, and electro-formation may be used to encapsulate and/or co-encapsulate nucleic acids and/or proteins with unique biological function into various degradable and non-degradable polymersomes. In other embodiments, a progressive saturation protocol may be used to prepare such polymersome encapsulations and/or co-encapsulations of proteins and/or nucleic acids.

Specifically, a progressive saturation protocol involves heating equal amounts of polymer (e.g. 10 mg) and PEG (e.g. 10 mg) at around 95° C. for around 1 h. The sample mixture may be centrifuged and cooled to room temperature. A solution of the product to be encapsulated (e.g., a gene editing system/composition containing protein and/or nucleic acid) may be prepared, such as in polybutylene succinate (PBS) at a pH of 7.4 pH. A small amount of the solution may first be added to the sample mixture (e.g. 10 μL), and mixed thoroughly followed by sonication at room temperature for around 30 min. The sample may be further diluted with a number of dilution steps. Specifically, each dilution step may involve addition of a volume of the solution containing the protein and/or nucleic acid, followed by thorough mixing and sonication at room temperature for around 30 minutes. After the dilution steps, the resulting sample may be dialyzed in isosmotic PBS for at least 30 h at around 4° C., employing at least a 1000 kDa molecular weight cutoff membrane. Surface attached product may be removed by proteolysis, via treatment with 0.4 wt % pronase solution. In various embodiments, encapsulation of the resulting polymersome suspension may be measured before and after proteolysis. Specifically, concentration of protein and/or nucleic acids may be measured using inductively coupled plasma optical emission spectroscopy (ICP-OES), inductively coupled plasma mass spectrometry (ICP-MS), matrix assisted absorption mass spectrometry time of flight (MALDI-TOF), atomic absorption spectroscopy (AAS) and/or UV-Vis absorption spectroscopy.

The progressive saturation steps may provide favorable results for encapsulating proteins and/or nucleic acids as gene editing systems within polymersomes. That is, the loading capacity of the gene editing composition may be significantly improved compared to polymersome encapsulation that uses other techniques (e.g., direct hydration, etc.). Without wishing to be bound to a particular theory, such improvements suggest that the polymersome formation process is not complete during the initial dilution step, and that further encapsulation is accomplished with each subsequent addition of protein solution. Specific progressive saturation protocols may be developed for specific gene editing compositions (e.g., proteins and/or nucleic acids) and polymersome types by optimizing and combining various steps from multiple liposome formation methods. Factors influencing the final concentrations of the gene editing systems, the relative loading levels that can be achieved within the polymersome carrier (i.e., w/w% composition/polymer), and the efficiency of gene editing composition encapsulation may be systematically evaluated. Factors such as the molecular weight of the polymer, the properties of the gene editing composition, the pH and nature of the buffered solution, the exact polymer hydration conditions (i.e., time, temperature, and blending technique), the number and duration of sonication steps, and the addition or avoidance of freeze-thaw cycles may all have effects on the concentration and the fidelity of the final polymersome-encapsulated gene editing system.

In some embodiments, there may be a direct tradeoff between encapsulation efficiency and the final loading capacity (i.e., weight percentage of composition to polymer) that can be achieved based on the concentration of gene editing composition used for each dilution step. Aqueous encapsulation of protein and/or nucleic acid is preferred to surface-associated compositions in order to assure that the final product meets the objectives for utilizing a polymersome delivery vehicle—that is, to improve biochemical stability, to increase circulatory half-life, to minimize adverse side effects, and to achieve controlled release of the associated protein. The various embodiment techniques may be employed using different gene editing compositions that vary over a large range of molecular weights and sizes.

Creation of various polymersome encapsulations of model proteins and model nucleic acids, as well as co-encapsulations of such proteins and nucleic acids, may be created using conventional techniques as well as progressive saturation. For example, myoglobin (Mb; Mw=around 17 kDa), which has a size and thermal stability (i.e., denaturation above 60° C.) comparable to other small proteins with therapeutic potential, was used as a model protein. Myoglobin also has a strong ultraviolet (UV) absorbance that enables ready identification of its functional status, as determined by the redox state of its iron-containing heme group. Other model proteins that may be used in such encapsulations are bovine serum albumin (BSA; Mw=around 66 kDa) and catalase (Mw=around 250 kDa). The encapsulation and co-encapsulation of model proteins having various sizes provides a range of sizes of functional proteins that may be used in various embodiments. Further, various DNA plasmids may be used as model nucleic acids for polymersome encapsulations, such as plasmid DNA encoding the mammalian expression vector for expression of green fluorescent protein (GFP) using the elongation factor 1 alpha (EF1a) promoter) (i.e., pEF-GFP DNA). The pEF-GFP DNA is around 5000 base-pairs, and has a molecular weight of around 3283 kDa.

The various embodiments may be prepared using any of a variety of amphiphilic polymers comprised of PEG and a hydrophobic block that is a biodegradable polymer (e.g., a biodegradable polyester, poly(amide), poly(peptide), poly(nucleic acid), etc.). Examples of biodegradable polymers that may form the hydrophobic block include, but are not limited to, poly(lactic acid), poly(glycolic acid), poly(lactic-co-glycolic acid), poly(caprolactone), poly(methyl caprolactone), poly(hydroxybutyrate), poly(hydroxyvalerate), poly(hdyroxyhexanoate), poly(hydroxyoxtanoate), and poly(trimethylene carbonate).

### Experimental

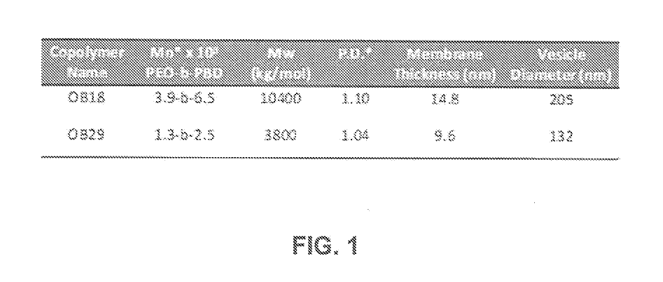

Different polymersome formulations may be used to compare the encapsulation properties between resulting particles and the techniques for formation. For example, PEO-b-PBD diblock copolymers are used to form polymersomes that possess fully PEGylated surfaces. Such surfaces, being uncharged and non-degradable, provide an ideal system for ensuring vesicle integrity and minimizing unwanted protein interactions or modifications. Two different molecular weight PEO-b-PBD diblock copolymers, “OB18” and “OB29”, are employed to determine the generalizability of the results as they pertain to polymersomes of different minimal sizes, PEG lengths, and membrane core thicknesses. FIG. 1 provides a table showing a comparison of various properties of OB18 and OB29.

Comparative and quantitative studies were performed as follows.

Materials

PEO(3900)-b-PBD(6500) (OB18) and PEO(1300)-b-PBD(2500) (OB29) were purchased from Polymer Source (Dorval, Quebec, Canada). Horse skeletal muscle Mb, bovine serum albumin (BSA), catalase (C), sodium hydrosulfite, poly(ethylene glycol) dimethyl ether (PEG; Mn=˜500), protease from Streptomyces griseus (“pronase”), and dichloromethane (DCM) were purchased from Sigma-Aldrich (St. Louis, USA). Dialysis tubing and vials were purchased from Spectrum Laboratories (Rancho Dominguez, USA). Other chemicals for conventional use were purchased from Fisher Scientific (Suwanee, USA). All chemicals were of reagent grade unless otherwise stated.

The particle sizes were measured using Delsa™ Nano, a dynamic light scattering (DLS) instrument (Beckman Coulter, Indianapolis, USA). Myoglobin concentrations were determined by optical absorption spectroscopy using a Genesys™ 10S UV-Vis spectrophotometer (Thermo Scientific, Suwanee, USA). BSA concentrations in polymersome-encapsulated suspensions were measured using a Micro Bicinchoninic acid (micro-BCA) Protein Assay Kit, utilizing UV-Vis spectrophotometry and by following the manufacturer's protocols (Pierce Biotechnology, Inc; Rockford, Ill., USA). DNA concentrations in polymersome-encapsulated suspensions were determined using a Vista-PRO CCD ICP-OES (Varian, USA).

Specifically, in order to quantify DNA encapsulation, a DNA sample was first reacted with the platinum(II)-based agent cisplatin, and a standard curve was generated showing how the DNA concentration correlated to the amount of DNA-bound platinum in suspension. Such standard curve was created by measuring, following serial dilutions of the DNA sample, the concentration of DNA using a NanoDrop™ spectrophotometer (i.e., UV-vis spectrophotometry) and the concentration of platinum by ICP-OES. In this manner, the amount of DNA in quantitative studies of polymersome-encapsulated DNA suspensions was calculated through disruption of the formed polymersomes, via addition of the surfactant Tween80 followed by measurements of the platinum concentration (and hence DNA concentration in suspension) via ICP-OES. This process avoided using the same measurement technique (i.e., UV-vis spectrophotometry) for both protein and DNA, which would, otherwise, interfere with accurate measurements.

### EXAMPLES

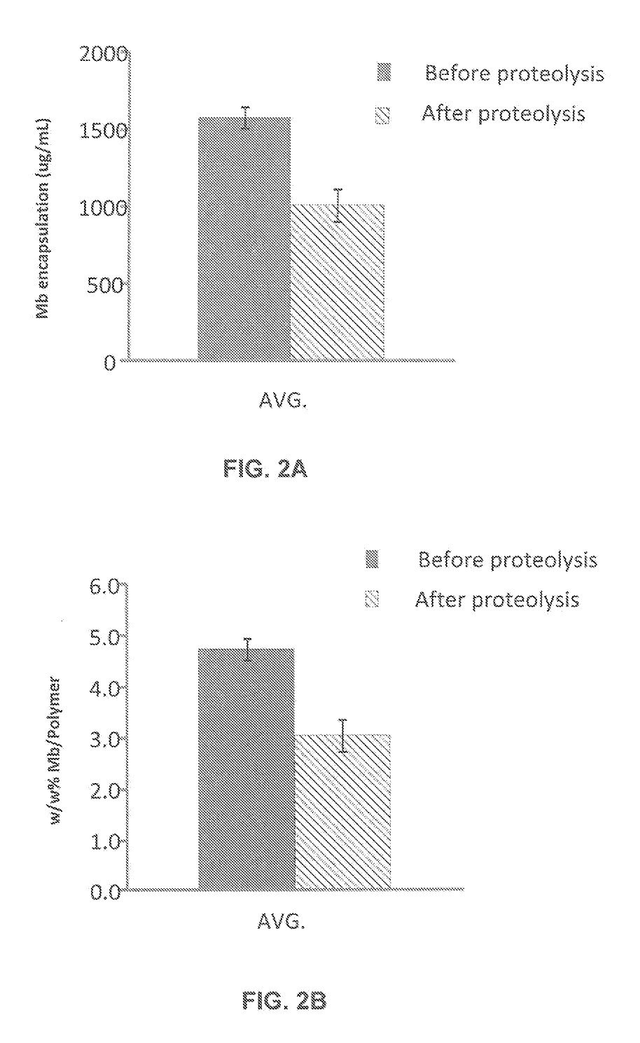

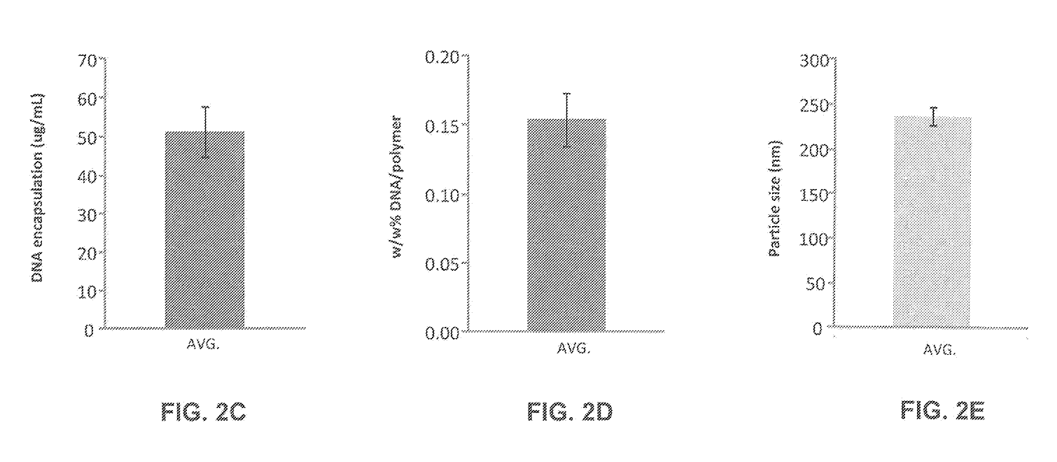

In a first comparison model, myoglobin (Mb) and pEF-GFP DNA were co-encapsulated in OB29 using the progressive saturation method. FIG. 2A shows the encapsulation amount of Mb (μg/mL), before and after proteolysis for 18 hours, which is a technique utilized to remove any non-specifically bound (i.e. surface-associated) protein from the polymersome suspensions. The amount of encapsulated Mb in the final polymersomes was quantified using UV-Vis absorption spectroscopy (also referred to as spectrophotometry).

FIG. 2B shows the average loading capacity as the average final weight percentage of protein-to-polymer (i.e., w/w % Mb/polymer) in the first comparison model, before and after proteolysis. FIG. 2C shows the average encapsulation amount of the DNA (μg/mL) in nanoscale polymersomes generated using the first comparison model, before and after proteolysis. The encapsulation amount in FIG. 2C was quantified using ICP-OES to measure DNA-bound platinum in solution after vesicle disruption with Tween80. FIG. 2D shows the average loading capacity as the average final weight percentage of DNA-to-polymer (i.e., w/w % DNA/polymer) in the resulting polymersomes, before and after proteolysis. FIG. 2E show the average size (i.e., hydrodynamic diameter) of polymersomes that contain both Mb and DNA within their aqueous cavities and that were generated in the first comparison model. As shown, an encapsulation amount of around 1-1.5 mg/mL for Mb (all contained within the aqueous cavities of the polymersomes) was achieved using the first comparison model, corresponding to around 3-4 wt % of the final polymersome composition. Further, analysis for DNA was achieved using the first comparison model, corresponding to around 0.15 wt % of the final polymersome composition, which displayed a size measuring about 200 nm in diameter.

In a second comparison model, Mb and pEF-GFP DNA were co-encapsulated in OB29-based polymersomes using the thin film hydration method. Thin film hydration and direct hydration are conventional procedures for encapsulating water-soluble species within the aqueous cavities of polymersomes and have been described in a variety of publications (e.g., O'Neil et al., A Novel Method for the Encapsulation of Biomolecules into Polymersomes via Direct Hydration, Langmuir 2009 25 (16), 9025-9029).

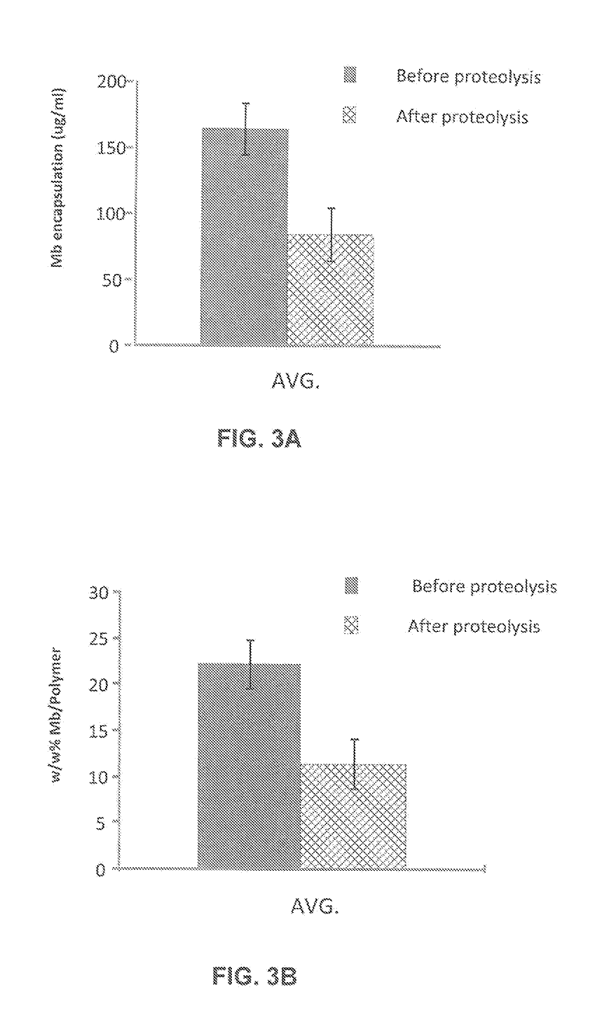

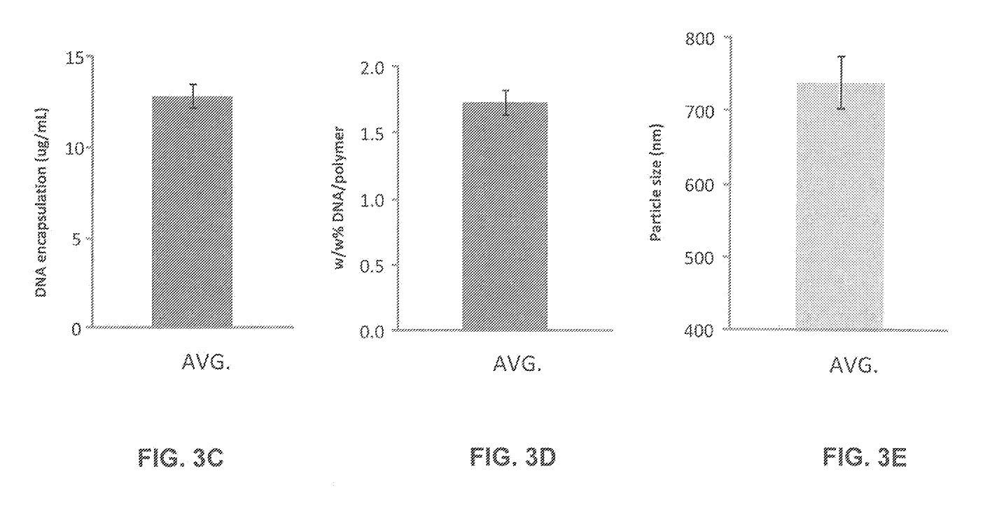

FIG. 3A shows the encapsulation amount of Mb (μg/mL) in the nanoparticles generated using the second comparison model before and after proteolysis for 18 hours. The encapsulation amount was quantified using UV-Vis absorption spectroscopy.

FIG. 3B shows the average loading capacity as the average final weight percentage of protein-to-polymer (i.e., w/w % Mb/polymer) for polymersomes that encapsulated both Mb and DNA within the same nanoscale vesicle construct and that were generated in the second comparison model, before and after proteolysis. FIG. 3C shows the average encapsulation amount of the DNA (μg/mL) in the polymersomes, before and after proteolysis. The encapsulation amount in FIG. 3C was quantified using ICP-OES of DNA-bound platinum. FIG. 3D shows the average loading capacity as the average final weight percentage of DNA-to-polymer (i.e., w/w % DNA/polymer) in the polymersomes generated in the second comparison model, before and after proteolysis. FIG. 3E show the average size (i.e., diameter) of polymersomes that encapsulated both Mb and DNA within the same nanoscale vesicle construct. As shown, an encapsulation of around 80 μg/mL for Mb was achieved using the second comparison model, corresponding to around 10 wt % of protein in the final polymersome formulation. Further, an encapsulation of around 12 μg/mL for DNA was achieved using the second comparison model, corresponding to around 0.18 wt % of the final polymersome formulation. The final polymersome formulation that was generated using the second comparison model and that encapsulated both Mb and DNA within the same nanoscale vesicle construct was found to have a mean particle size of about 730 nm in diameter.

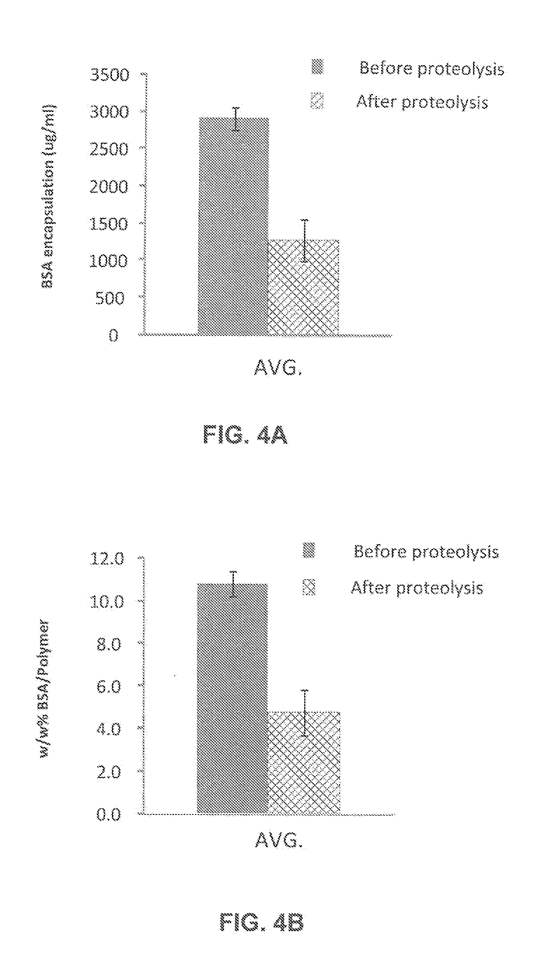

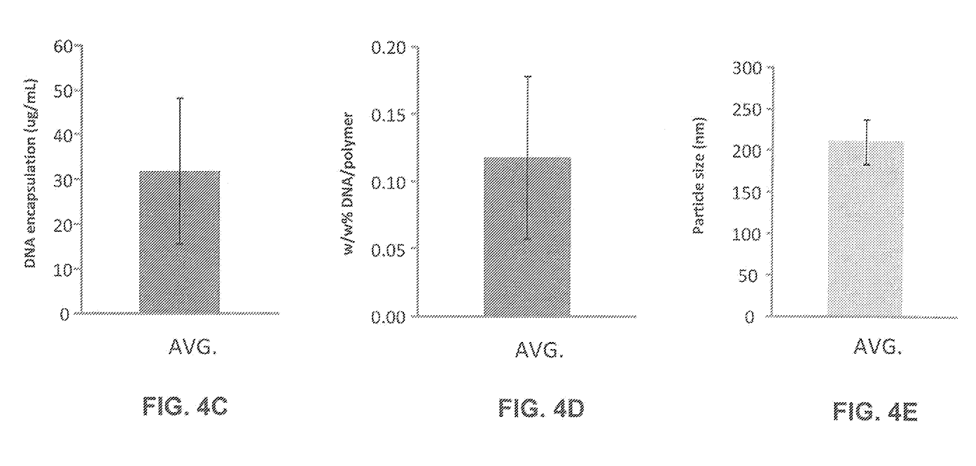

In a third comparison model, BSA and pEF-GFP DNA were co-encapsulated in OB29-based polymersomes using the progressive saturation technique. FIG. 4A shows the average encapsulation amount of BSA (μg/mL) in the resulting polymersomes, before and after proteolysis for 18 hours. The amount of encapsulated protein was quantified using the micro-BCA assay.

FIG. 4B shows the average loading capacity as the average final weight percentage of protein-to-polymer (i.e., w/w % BSA/polymer) in the polymersomes generated in the third comparison model, before and after proteolysis. FIG. 4C shows the average encapsulation amount of the DNA (μg/mL) in the polymersomes, before and after proteolysis. The encapsulation amount in FIG. 4C was quantified using ICP-OES of DNA-bound platinum. FIG. 4D shows the average loading capacity as the final weight percentage of DNA-to-polymer (i.e., w/w % DNA/polymer) in the polymersomes generated in the third comparison model, before and after proteolysis. FIG. 4E show the average size (i.e., diameter) of polymersomes that encapsulated both BSA and DNA within the same nanoscale vesicle construct. As shown, encapsulation of around 1-1.5 mg/mL for BSA was achieved using the third comparison model, corresponding to around 3-4 wt % of the final polymersomes formulation. Further, encapsulation of around 50 μg/mL for DNA was achieved using the third comparison model, corresponding to around 0.15 wt % of the final polymersome formulation. Polymersomes that encapsulated both BSA and DNA within the same nanoscale vesicle construct had a size of about 200 nm in diameter when generated in the third comparison model.

As shown by comparing the results shown in FIGS. 2E, 3E and 4E to the nanoparticle sizes in FIG. 1, the final polymersomes were not made significantly larger by the co-encapsulation of protein (either Mb or BSA) with nucleic acid (pEF-GFP DNA) using the progressive saturation technique. However, when generated using thin film hydration, the sizes of final polymersomes that encapsulated both protein and nucleic acid within the same nanoscale vesicle construct were much larger.

Further polymersome encapsulations using the same protocols have been and continue to be developed as comparison models using polymersomes made of other non-degradable polymers (e.g., OB18), as well as those made of various degradable polymers. Examples of such degradable polymers include, but are not limited to: PEO(5000)-b-PCL(16300) (“P2350-EOCL”); PEO(2000)-b-PMCL(11900) (“OCL”); PEO(2000)-b-PMCL(8300) (“OMCL”); PEO(1100)-b-PTMC(5100) (“OTMC”); and PEO(2000)-b-PTMC/PCL(11200) (“OTCL”).

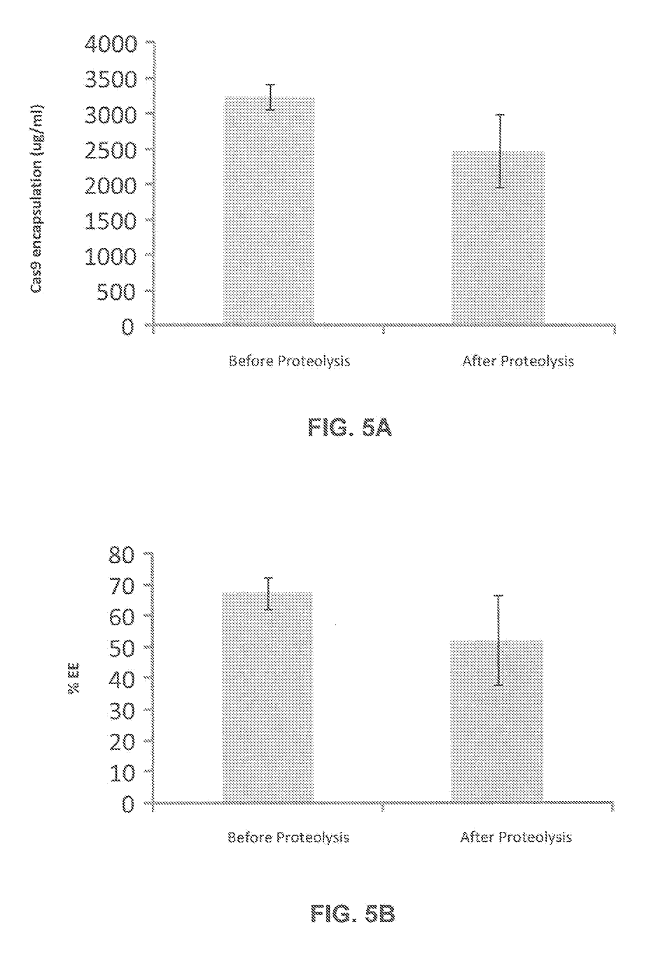

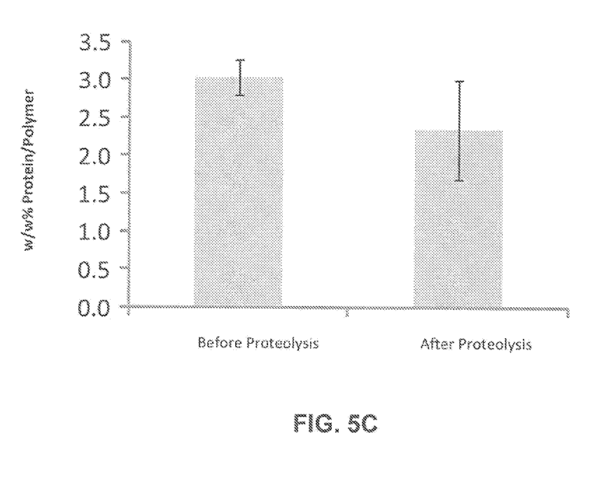

An example embodiment of polymersome-encapsulated RNA-directed nuclease was prepared using Cas9 protein derived from Streptococcus pyogenes. Specifically, non-degradable polymers of 4-hydroxy benzoic ester Poly(butadiene2500-b-ethylene oxide1300) (“OB29-Bz”) were used t o encapsulate Cas9 protein (powder, 250 μg/vial) using the progressive saturation protocol in which the sample was dialyzed (MW cutoff=100 kDa) for 30 h at room temperature. FIG. 5A shows the average encapsulation amount of Cas9 protein (μg/mL) in the resulting polymersomes, before and after proteolysis. The encapsulation was quantified using the micro-BCA assay. FIG. 5B shows the average encapsulation efficiency of Cas9 protein (% EE) in the resulting polymersomes, before and after proteolysis. FIG. 5C shows the average loading capacity as the average final weight percentage of protein-to-polymer (i.e., w/w % Cas9 protein/polymer) in the polymersomes generated in this example embodiment, before and after proteolysis. As shown, an encapsulation of around 3 mg/mL for Cas9 protein was achieved in this example embodiment, corresponding to around 3-4 wt % of the final polymersome formulation. The encapsulation efficiency for Cas9 protein was around 65%.

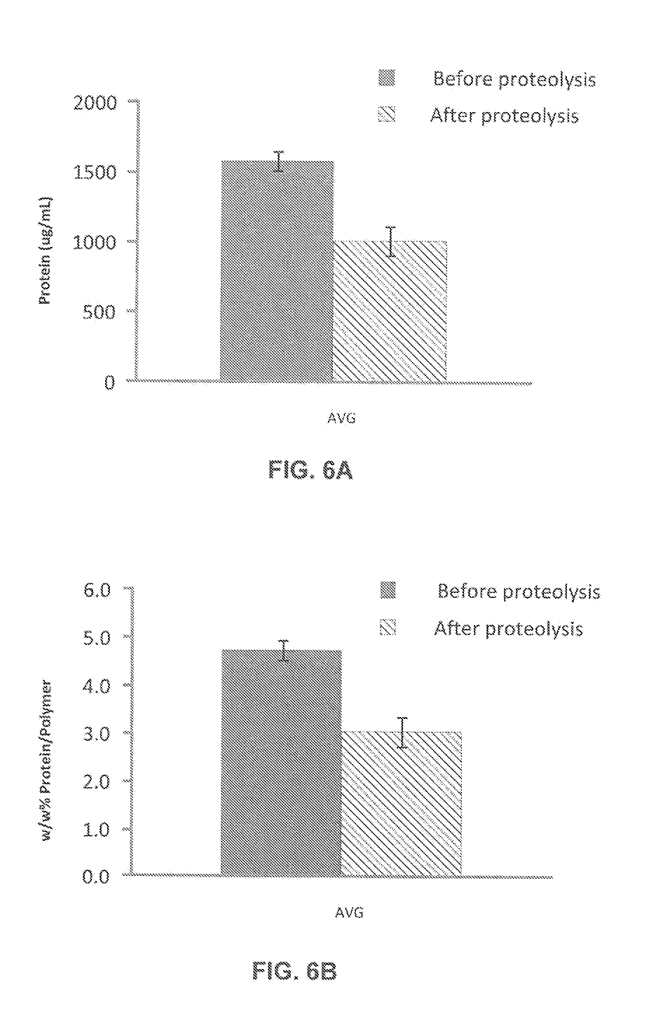

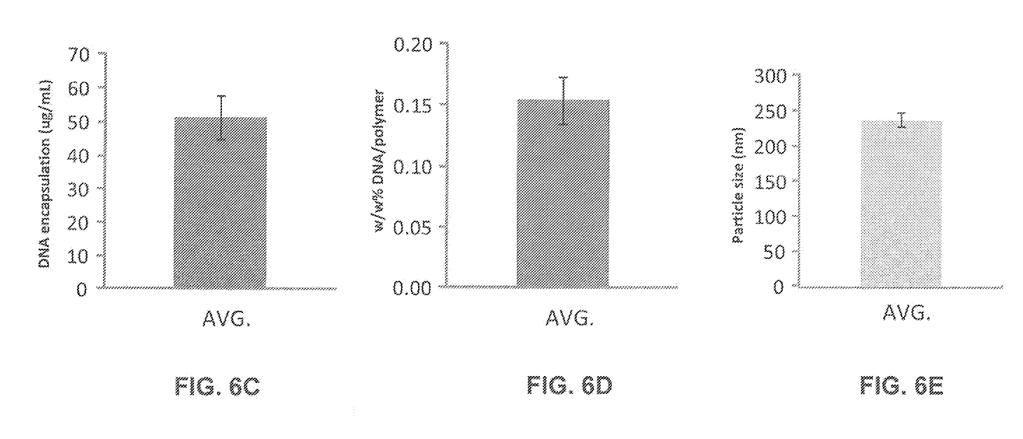

In a fourth comparison model, Cas9 protein and pEF-GFP DNA were co-encapsulated in OB29-Bz using the progressive saturation technique. FIG. 6A shows the average amount of Cas9 protein (μg/mL) that was encapsulated in the polymersomes generated in the fourth comparison model. The encapsulation amount was quantified by the micro-BCA assay. FIG. 6B shows the average loading capacity as the average final weight percentage of protein-to-polymer (i.e., w/w % Cas9 protein/polymer) in the polymersomes, before and after proteolysis.

FIG. 6C shows the average amount of DNA (μg/mL) encapsulated in the nanoparticles generated in the fourth comparison model, before and after proteolysis. The encapsulation amount in FIG. 6C was quantified using ICP-OES of DNA-bound platinum. FIG. 6D shows the average loading capacity as the average final weight percentage of DNA-to-polymer (i.e., w/w % DNA/polymer) in the resulting polymersomes, before and after proteolysis. FIG. 6E show the average size (i.e., diameter) of the final polymersomes generated in the fourth comparison model. As shown, encapsulation of around 1 mg/mL for Cas9 protein was achieved using the fourth comparison model, corresponding to around 3 wt % of the final polymersome formulation. Further, an encapsulation of around 50 μg/mL was achieved for DNA using the fourth comparison model, corresponding to around 0.15 wt % of the final polymersome formulation that encapsulated both Cas9 protein and DNA within the same nanoscale vesicle construct. These polymersomes were about 220 nm in diameter.

What is claimed is:

1. A composition for genetic modification, comprising:
a synthetic polymer vesicle; and
a gene editing system encapsulated in the synthetic polymer vesicle, the gene editing system comprising a nucleic acid component configured to interact with a target sequence in a host cell genome.

2. The composition of claim 1, wherein the gene editing system further comprises a protein component.

3. The composition of claim 2, wherein:
the protein component comprises a ribonucleic acid (RNA)-directed nuclease; and
the nucleic acid component comprises a guide RNA that is complementary to the target sequence.

4. The composition of claim 3, wherein:
the nucleic acid component further comprises an exogenous deoxyribonucleic acid (DNA) repair template;
the RNA-directed nuclease is configured to create a double stranded break in the host cell genome adjacent to the target sequence; and
a repair process in the host cell triggers modification of the host cell genome based on the exogenous DNA repair template, during re-ligation of the host cell genome.

5. The composition of claim 4, wherein the DNA repair template comprises end regions that are homologous to regions of the host cell genome flanking the double stranded break induced by the RNA-directed nuclease.

6. The composition of claim 1, wherein the gene editing system is included in the synthetic polymer vesicle in an amount of at least 3% by weight relative to the total weight of the composition.

7. The composition of claim 1, wherein the protein component comprises an enzyme in native form or a messenger RNA (mRNA) molecule configured to be translated into an enzyme after delivery into the host cell.

8. The composition of claim 2, wherein the protein component is delivered as an expression vector containing a deoxyribonucleic acid (DNA) sequence encoding an enzyme.

9. The composition of claim 8, wherein the nucleic acid component comprises a DNA sequence encoding a guide ribonucleic acid (RNA).

10. The composition of claim 9, wherein the DNA sequence encoding the enzyme and the guide RNA are provided on a single expression vector.

11. The composition of claim 2, wherein the protein component comprises an enzyme configured to cut the host genome based on binding of the nucleic acid component to a complementary segment of the host genome.

12. The composition of claim 11, wherein the enzyme comprises Clustered Regularly Interspaced Short Palindromic Repeats (CRISPR) associated protein 9 (Cas9).

13. The composition of claim 2, wherein:
the nucleic acid component comprises an expression vector that includes a transposon; and
the protein component comprises a transposase.

14. The composition of claim 13, wherein the transposase is one of:
a native enzyme;
a messenger ribonucleic acid (mRNA) molecule that is configured to be translated into the enzyme after delivery into the host cell; and
a deoxyribonucleic acid (DNA) sequence encoding the enzyme.

15. The composition of claim 14, wherein the DNA sequence is provided on the expression vector that includes the transposon.

16. The composition of claim 1, wherein the synthetic polymer vesicle is generated from at least one block copolymer comprising:
a hydrophilic block that includes poly(ethylene oxide); and
a hydrophobic block.

17. The composition of claim 15, wherein the hydrophobic block is selected from aliphatic poly(anhydrides), poly(nucleic acids), poly(esters), poly(ortho esters), poly(peptides), poly(phosphazenes) and poly(saccharides).

18. The composition of claim 15, wherein the hydrophobic block comprises one or more of poly(lactide) (PLA), poly(glycolide) (PLGA), poly(lactic-co-glycolic acid) (PLGA), poly(ε-caprolactone) (PCL), or poly (trimethylene carbonate) (PTMC).

19. A method of modifying a host cell genome, the method comprising:
encapsulating, in a polymersome, a gene editing system that comprises:
a protein component; and
a nucleic acid component configured to interact with a target nucleic acid sequence in the host cell; and
delivering the encapsulated gene editing system to the host cell,
wherein the polymersome is configured to selectively release the gene editing system in the host cell.

20. The method of claim 19, wherein delivering the encapsulated gene editing system to the host cell comprises administering to a subject an effective amount of a composition containing the encapsulated gene editing system.

21. The method of claim 19, wherein the encapsulated gene editing system is prepared using a progressive saturation protocol.

22. A method of manufacturing a suspension of an encapsulated gene editing composition, the method comprising:
thermally blending a quantity of a block copolymer with a quantity of a low molecular weight polyethylene glycol (PEG) to create a PEG/polymer formulation;
adding an aliquot of a solution of the gene editing composition to a sample containing the PEG/polymer formulation; and
performing at least one dilution step such that polymersomes that are generated are progressively saturated with the gene editing composition,
wherein the gene editing composition comprises:
a protein component; and
a nucleic acid component configured to interact with a target sequence in a host cell genome.

23. The method of claim 22, wherein the block copolymer comprises an amphiphilic diblock copolymer.

24. The method of claim 23, wherein the amphiphilic diblock copolymer comprises poly(ethylene oxide)-block-poly(butadiene) (PEO-b-PBD).

25. The method of claim 22, wherein the generated polymersomes have an encapsulation efficiency of at least 50% with respect to the gene editing composition.

26. A kit, comprising:
a pharmaceutical composition comprising a gene editing system encapsulated in a synthetic polymer vesicle, the gene editing system comprising:
a protein component; and
a nucleic acid component configured to interact with a target sequence in a host cell genome; and
an implement for administering the pharmaceutical composition intravenously, via inhalation, topically, per rectum, per the vagina, transdermally, subcutaneously, intraperitoneally, intrathecally, intramuscularly, or orally.
